(19) United States
(12) Patent Application Publication
(10) Pub. No.: US 2019/0290145 A1
(43) Pub. Date: Sep. 26, 2019

(54) SYSTEM AND METHOD FOR REMOTE DETECTION OF CARDIAC CONDITION

(71) Applicant: CONTINUSE BIOMETRICS LTD., Tel Aviv (IL)

(72) Inventors: Zeev ZALEVSKY, Rosh HaAyin (IL); Sagi POLANI, Tel-Aviv (IL); Javier GARCIA, Valencia (ES)

(21) Appl. No.: 16/316,415

(22) PCT Filed: Jul. 12, 2017
(86) PCT No.: PCT/IL2017/050792

§ 371 Date: Jan. 9, 2019

Related U.S. Application Data
(60) Provisional application No. 62/362,189, filed on Jul. 14, 2016.

(51) Int. Cl. A61B 5/024; A61B 7/04; A61B 5/00; A61B 5/0245

(52) U.S. Cl. CPC A61B 5/02416; A61B 5/0245; A61B 5/002; A61B 7/04

(57) ABSTRACT

A monitoring system is presented, configured for use in monitoring heart condition of a subject. The system comprises: a receiver utility for receiving input data comprising data corresponding to signals continuously collected from the subject over time N and being indicative of a sequence of heart sounds; and a processing utility configured and operable for processing said input data to determine data indicative of heart condition of the subject. The processing comprises: identifying within said input data a sequence of acoustic signals corresponding to heart sounds comprising S1 signals corresponding to ventricular contraction and S2 signals corresponding to end of ventricular systole; analyzing the sequence of acoustic signals in accordance with parameters including signal periodicity, amplitude of S1 and S2 sounds, and intervals between S1 and S2 sounds within the collected sequence; and generating data indicative of cardiac condition of the subject in accordance with the analyzed data.

### TECHNOLOGICAL FIELD

The present invention is in the field of monitoring of parameters of a subject and may be relevant for remote monitoring of a subject's cardiac condition.

### BACKGROUND ART

References considered to be relevant as background to the presently disclosed subject matter are listed below:

Bilchick K C et al. 2002. Prognostic value of heart rate variability in chronic congestive heart failure (Veterans Affairs' Survival Trial of Antiarrhythmic Therapy in Congestive Heart Failure). American Journal of Cardiology, 90(1): 24-28.

Covic A and Siripol D. 2015. Pulse Wave Velocity Ratio: The New “Gold Standard” for Measuring Arterial Stiffness. Hypertension, 65: 289-290.

Hosenpud, J. D., & Greenberg, B. H. (2007). Congestive heart failure (3rd ed.). Philadelphia: Lippincott Williams & Wilkins.

Jackson C E et al. 2015. Differing Prognostic Value of Pulse Pressure in Patients With Heart Failure With Reduced or Preserved Ejection Fraction. Eur Heart J, 36(18): 1106-1114.

Mitchell G F et al. 2001. Fourth Workshop on Structure and Function of Large Arteries: Part III—Pulsatile Hemodynamics in Congestive Heart Failure. Hypertension, 38: 1433-1439.

Peacock W F, Harrison A and Maisel A S. 2006. The Utility of Heart Sounds and Systolic Intervals Across the Care Continuum. Congestive Heart Failure, 12 (Suppl 1): 2-7.

Roger V L. 2013. Heart Failure Compendium: Epidemiology of Heart Failure. Circulation Research, 113: 646-659.

Wang S et al. 2013. Acoustic cardiography helps to identify heart failure and its phenotypes. Int J Cardiol, 10; 167(3): 681-6.

Yancy C W et al. 2013. ACCF/AHA Practice Guideline for the Management of Heart Failure: A Report of the American College of Cardiology Foundation/American Heart Association Task Force on Practice Guidelines. Circulation, 128: e240-e327.

Ziaeian B and Fonarow G C. 2016. Epidemiology and Aetiology of Heart Failure. Nature Review Cardiology, 13: 368-378.

Acknowledgement of the above references herein is not to be inferred as meaning that these are in any way relevant to the patentability of the presently disclosed subject matter.

### BACKGROUND

Congestive heart failure (CHF) is a heart condition affecting over 5 million people in the United States and over 37 million individuals globally. CHF occurs when the heart is incapable of efficiently pumping blood at sufficient rate to meet the body's need for supply of oxygen and nutrition. Efficient monitoring of patient's cardiac conditions may be used to simplify diagnostics and provide treatment that can assist patients in managing their condition effectively.

In various fields of medicine, patient's condition may be diagnosed with assistance of acoustic data collected from the patient. The commonly used stethoscope has become a symbol of a physician and provides detection of acoustic signals in a simple and non-invasive fashion. Additional techniques are being developed, providing electronic remote detection acoustic signals.

Several optical sensing techniques are known, which may be used for detection vibrations, and accordingly enable detection of acoustic data. For example:

U.S. Pat. No. 8,638,991 presents a method for imaging an object. The method comprises imaging a coherent speckle pattern propagating from an object, using an imaging system being focused on a plane displaced from the object.

US 2013/0144137 and US 2014/0148658 present a system and method for use in monitoring one or more conditions of a subject's body. The system includes a control unit which includes an input port for receiving image data, a memory utility, and a processor utility. The image data is indicative of data measured by a pixel detector array and is in the form of a sequence of speckle patterns generated by a portion of the subject's body in response to illumination thereof by coherent light according to a certain sampling time pattern. The memory utility stores one or more predetermined models, the model comprising data indicative of a relation between one or more measurable parameters and one or more conditions of the subject's body. The processor utility is configured and operable for processing the image data to determine one or more corresponding body conditions; and generating output data indicative of the corresponding body conditions.

### GENERAL DESCRIPTION

There is a need in the art for a novel technique enabling fast and continuous monitoring of cardiac activity of subjects/patients. The present invention provides system and method configured for remote monitoring of cardiac activity while generally not requiring physical contact with the monitored subject.

The technique of the present invention utilizes remote detection of vibration signals collected from one or more selected regions on the subject's body for determining data indicative of heart condition of the patient. The signal may typically be collected by detection of secondary speckle pattern formed from scattering and reflection of coherent electromagnetic radiation (e.g. optical radiation) returning from the one or more selected regions on the subject, and determining correlation between detected speckle patterns along certain time period using a selected sampling rate. The correlation function between consecutively detected speckle patterns correspond to variations of orientation, curvature and/or location of the inspected region, thereby enabling to detect nanometric scale movements at sampling rate determined by the collection and detection unit. Thus, the so-called speckle based detection technique enables detection of movements and/or vibrations associated with physical movement (internal or external) as well as acoustic signals causing material of the inspection region to vibrate as a membrane.

The technique of the present invention thus provides a monitoring system configured to include, or be connectable to, one or more speckle-based detection units. The monitoring system comprising a receiver unit configured and operable for receiving input data comprising data corresponding to signals collected from a subject at a selected sampling rate over time, and a processing utility configured for receiving said input data indicative of the collected signals and for processing said data to determine data indicative of heart condition of the subject.

The processing utility is configured and operable for identifying, within the input data, one or more data sequences indicative of acoustic signals and corresponding to heart sounds. Such heart sound signals generally comprise signals corresponding to S1 sounds associated with ventricular contraction and signals corresponding to S2 sounds associated with end of ventricular systole, and may further comprise additional heart sounds such as S3 and/or S4. The processing utility further analyzes the sequence of acoustic signals in accordance with parameters including signal periodicity, amplitude of the detected acoustic signals (e.g. S1 and S2 sounds), energy of the acoustic signals (e.g. S2 sounds) and intervals between acoustic signals associated with S1 and S2 sounds within the collected sequence. The processing utility is further configured for generating data indicative of cardiac condition of the subject in accordance with the analyzed data. In this connection, the monitoring system of the present invention is configured for receiving input data in the form of one or more data streams (e.g. image data streams, or data about determined correlations between sequences of speckle patterns), comprising data associated with vibrations detected from one or more inspection regions on the subject/patient. Such vibrations may be at least partially associated with acoustic signals, such as heart sounds, breathing sounds, joint movement related sounds etc., originating from the subject's body. The technique of the present invention utilizes processing of the vibration signals, identifying acoustic signals associated with cardiac activity and analyzing the heart-related acoustic signals to determine data about heart condition of a subject/patient and generate corresponding output data.

Generally the analysis of heart sounds is based on detection of S1 and S2 sounds, corresponding respectively to ventricular contractions pushing blood into the aorta and to closure of the aortic and pulmonic valves at the end of ventricular systole, and determining cardiac related parameters such as heart rate, time difference between S1 and S2 sounds, amplitude and/or energy of the sounds, existence of additional heart sounds such as S3 and S4 appearing at the end or beginning of a cycle and often indicate illness conditions. Typically, analysis of these heart sounds is performed by a physician while listening to the patient's heart using a stethoscope. The use of the technique of the present invention enables remote monitoring of heart condition, as well as maintaining and storing data about heart sounds for additional processing and objective monitoring of variation in the patient's condition. Further, the use of optical inspection enables seamless monitoring of the patient's condition while not requiring any physical contact for collection of data about the patient.

Thus, according to a broad aspect of the invention, there is provided a monitoring system for use in monitoring heart condition of a subject, the system comprising:

a receiver utility for receiving input data comprising data corresponding to signals continuously collected from the subject over time and being indicative of a sequence of heart sounds;

a processing utility configured and operable for processing said input data to determine data indicative of heart condition of the subject; said processing comprises: identifying within said input data a sequence of acoustic signals corresponding to heart sounds comprising S1 signals corresponding to ventricular contraction and S2 signals corresponding to end of ventricular systole; analyzing the sequence of acoustic signals in accordance with parameters including signal periodicity, amplitude of S1 and S2 sounds, and intervals between S1 and S2 sounds within the collected sequence; and generating data indicative of cardiac condition of the subject in accordance with the analyzed data.

Generally, said cardiac condition may relate to Congestive Heart Failure (CHF).

According to some embodiments, said analyzing may comprise at least one of:

analyzing the amplitude of the signal S1 and determining a measure of force of ventricular contraction and amount of ventricular pressure developed during systole for identifying a condition of decreased cardiac contractility,

analyzing the amplitude and energy of the signal S2 and determining a measure of amount of closing pressure in an aorta and pulmonary arteries for identifying a condition of increased arterial blood pressure; and analyzing the time interval between the signals S1 and S2 of the cycle and determining whether said time interval is shorter than a predetermined value for identifying a condition of systolic heart failure.

According to some embodiments, said processing may further comprise analyzing a time pattern of at least two of said cycles for identifying a condition of appearance of a signal associated with a third heart sound S3 appearing at an end of a cycles and being indicative of illness condition.

According to some embodiments, said processing may further comprise analyzing a time pattern of at least two of said cycles for identifying a condition of appearance of a signal associated with a fourth heart sound S4 appearing at a beginning of a cycle and being indicative of illness condition.

According to some embodiments, said receiver unit may be configured for signal communication with at least one measurement unit operable for continuously collect said acoustic from the subject over time and providing said corresponding data to be received by the receiver unit.

The measurement unit may comprise: at least one radiation source unit configured for generating coherent electromagnetic radiation of a selected frequency range and to direct the generated radiation towards a target; and at least one radiation detection unit associated with said at least one radiation source unit, the at least one radiation detection unit is configured for collecting radiation returning from said target and for generating data indicative of a secondary speckle pattern formed in said collected radiation.

Additionally or alternatively, the receiver unit may be configured and operable for signal communication with a plurality of two or more measurement units, said processing utility being configured and operable for receiving input data corresponding to data received from said plurality of measurement units and for performing blind source separation on said input data to thereby separate heart related acoustic signals from other signals detected by said plurality of measurement units.

According to some embodiments, said plurality of two or more measurement unit may be configured such that said plurality of measurement units are directed at a plurality of different inspection regions on said subject.

According to some embodiments, said at least one measurement unit may be configured to utilize coherent radiation at a selected frequency range being between 1 GHz and 3 THz. The selected frequency range may be between 2 GHz and 300 GHz.

According to one other broad aspect of the invention there is provided a system comprising:

at least one radiation source unit configured for generating coherent electromagnetic radiation of a selected frequency range and to direct the generated radiation towards a target;

at least one radiation detection unit associated with said at least one radiation source unit, the at least one radiation detection unit is configured for collecting radiation returning from said target and for generating data indicative of a secondary speckle pattern formed in said collected radiation; and

a control unit connectable to said at least one radiation source unit and said at least one radiation detection unit and configured for receiving input data comprising at least one sequence of image data pieces associated with detected secondary speckle patterns and for processing said input data to determine one or more parameters of the target.

According to some embodiments, said target may be a patient, said one or more parameters of the target may comprise one or more parameter of cardiac activity of said patient.

According to some embodiments, the control unit may comprise at least one processing utility; the processing utility comprises:

a correlation module configured and operable for receiving data about a sequence of input data pieces, each corresponding to a detected speckle pattern, and for processing the sequence of data pieces to determine correlation function between consecutive data pieces, said correlation function being indicative of variation in position of points within an inspected region;

a signal detection module configured for receiving data about variation in position of points within said inspected region and determining therefrom signal data corresponding to selected one or more parameters of the target.

According to some embodiments, the signal detection module may comprise:

frequency filtering module configured and operable for selectively filtering said data about variation in position of points within an inspected region and determine signal data associated with acoustic signals originated from said inspected region;

cardiac activity module configured and operable for receiving said acoustic signals and determined signal portions associated with cardiac activity sounds;

cardiac efficiency module configured and operable for receiving data about cardiac activity sound from the cardiac activity module and determine one or more parameters of efficiency of cardiac activity.

According to some embodiments, said one or more parameters of efficiency of cardiac activity may comprise at least one of the following: heart rate, time interval between ventricular and atrial contraction, S3 and S4 cardiac sounds.

According to some embodiments, said selected frequency range of electromagnetic radiation may comprise radiation frequency between 1 GHz and 3 THz or between 3 GHz and 300 GHz. Said one or more parameters of the target may comprise data indicative of internal electrical activity of the target.

According to some embodiments, the system may comprise a plurality of radiation source units configured for directing electromagnetic radiation onto said target from a plurality of different direction and a corresponding plurality of radiation detection unit, said control unit being configured and operable for receiving input data comprising a plurality of data streams corresponding to secondary speckle patterns collected by said plurality of radiation detection units and for processing said input data by blind source separation to thereby determine tomographic data of said target.

Said control unit may further comprise at least one processing utility; the processing utility comprises:

a correlation module configured and operable for receiving input data about comprises a plurality of sequences of input data pieces, each corresponding to a detected speckle pattern, and for processing the sequence of data pieces to determine correlation function between consecutive data pieces, said correlation function being indicative of variation in position of points within an inspected region;

a blind source separation module configured and operable for receiving a plurality of data pieces each corresponding to correlations between consecutive speckle patterns detected by certain radiation collection unit and for processing said plurality of correlations for determining tomographic data indicative of parameters of the target.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 exemplifies schematically a monitoring system according to some embodiments of the invention;

FIG. 2 shows ECG, pressure and acoustic parameters associated with normal heart cycle and heart cycle of a patient with systolic heart failure;

FIG. 3 shows general ECG and acoustic parameters of heart activity;

FIG. 4 is a flow chart exemplifying operation of the present technique according to some embodiments;

FIG. 5 schematically illustrates a configuration of the monitoring system and processing utility thereof according to some embodiments of the invention;

FIG. 6 exemplifies schematically a monitoring system according to some embodiments of the invention utilizing a plurality of measurement units and providing blind source separation option; and

FIG. 7 exemplifies nano-vibration data collected using speckle based measurement unit in micro-wave electromagnetic radiation.

### DETAILED DESCRIPTION OF EMBODIMENTS

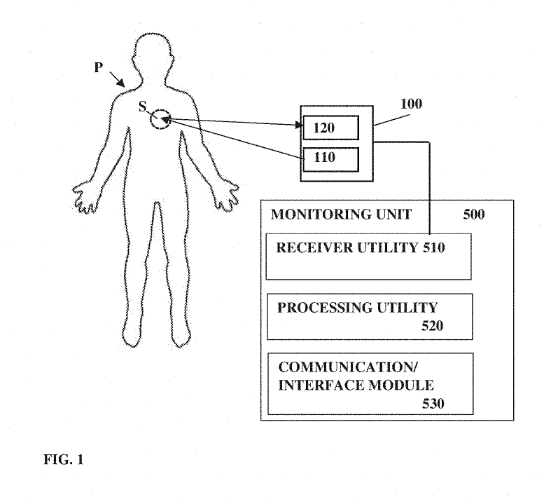

As indicated above, the technique of the present invention utilizes remote detection of vibration and acoustic signals for determining and providing data indicative of heart condition of a subject. Reference is made to FIG. 1 illustrating schematically a monitoring system 500 configured according to some embodiments of the present invention and connectable to one or more measurement units 100 (one such measurement unit is shown in this example) used for collecting data from an inspection region S on a subject P. The monitoring system 500 includes a receiver utility 510 connectable to the one or more measurements units 100 and a processing utility 520 configured for receiving input data indicative of vibrations detected at the inspection region and for processing the input data to determine heart condition of the subject. The monitoring system 500 generally also includes a communication/user interface module 530 and may also include memory utility and/or network communication utility which are not specifically shown here.

As indicated, the monitoring system 500 is connectable to one or more measurement units 100. The measurement units 100 may typically be speckle based measurement units. More specifically, such measurement unit 100 may generally include a radiation source unit 110 configured for generating coherent radiation of selected wavelength range and direct the generated radiation to an inspection region S on the subject P; and a detection unit 120 configured for collecting radiation returning/scattered from the inspection region S and generate image data pieces corresponding to secondary speckle pattern in the collected radiation. To this end the detection unit 120 generally acts to capture image data pieces corresponding to an intermediate plane along optical path of collected radiation practically providing defocused images of the inspection region S. This is provide either by utilizing an optical arrangement imaging a selected intermediate plane onto a detector array, or by location of the detector array at the selected intermediate plane. The detection unit 120 is operated at a selected sampling rate to provide image data sequence while each image data piece has certain geometrical resolution being equal or lower than geometrical resolution of the detector array. In some configurations, the detection unit 120 may also include one or more optical elements configured for increasing temporal sampling rate at a price of reducing geometrical resolution.

The receiver utility 510 of the monitoring system 500 is configured and operable for receiving a stream of input data indicative of vibration signals of the inspection region. Such input data may generally include a stream of image data pieces indicative of detected secondary speckle patterns collected at a predetermined, selected sampling rate. The receiver utility 510 may be configured for pre-processing the input data to determine correlation between consecutive image data pieces for determining vibration data. Alternatively or additionally, the one or more measurement units 100 may perform such pre-processing and the receiver utility 510 may receive data indicative of such correlation between different speckle patterns, where the correlation between collected speckle patterns corresponds to variation in location, orientation and/or curvature of the inspection region and thus when collected over time at sufficient sampling rate the correlation data corresponds to vibrations or movement of the inspection region.

The receiver utility 510 transfers the received input data to the processing utility 520 and/or to a memory utility for storage and further processing and analysis. The processing utility 520 may include one or more processor units and/or be connectable with remote one or more processors and is configured and operable for processing the input data to generate and provide data indicative of heart condition of the subject. More specifically, the processing utility 520 may operate for filtering the input data in accordance with signal frequency and amplitude to provide data associated with acoustic signals with reduced effects or noise resulting from mechanical vibrations. Further, the processing utility 520 may apply frequency filtering to the acoustic signals and locate repeating signal patterns corresponding to heart cycles (e.g. having 40-200 repeating cycles per minute). Within the repeating cycles, the processing utility 520 may operate to identify signal features associated with S1 and S2 heart sounds corresponding to general pumping activity of the subject's heart, and determine data about heart condition based on the acoustic signals relating to heart activity. Typically, in addition to the S1, S2 heart cycle, the processing utility may identify additional heart related acoustic data providing additional indications on heart conditions. For example, the processing utility may identify S3 and/or S4 heart sounds as well as identify breathing cycle that may also be associated with heart conditions.

Generally, according to some embodiments of the invention, the monitoring system 500 may be configured for monitoring and detecting conditions associated with Congestive heart failure (CHF) or other heart diseases. CHF is a heart condition affecting over 5 million people in the United States and over 37 million individuals globally, and is one of the leading causes of hospital admissions. CHF has several clinical manifestations, at the end-stage of the disease abnormal function of the heart leads to inadequate blood flow to fulfill the needs of the body's tissues. Typically, the heart loses propulsive power because the cardiac muscle loses capacity to stretch and contract. Often, the ventricles do not adequately fill with blood between heartbeats and the valves regulating blood flow may become leaky, allowing regurgitation or backflow of blood. The impairment of arterial circulation may deprive vital organs of oxygen and nutrients. CHF patients are more prone to experience respiratory distress, anxiety, stress, cardiac decompensation, fatigue, weakness inability to carry out daily tasks and venous thromboembolism. However, not all CHF patients suffer debilitating symptoms immediately. Some may live actively for years, with few limitations. However, the disease is relentlessly progressive, and as it progresses, it tends to become increasingly difficult to manage. Therefore, with appropriate tracking of the progression or regression of CHF, treatments could be managed more effectively. It should be noted that diagnosis of heart condition is performed by a physician based on various measurements of heart functionality and generally using Electrocardiogram (ECG) and echocardiogram. However, monitoring of heart sounds using the technique of the present invention provides data on initial signs of heart condition, similarly to the use of stethoscope by a physician, as well as enable long time monitoring while requiring limited and preferably no physical contact with the patient and enabling objective monitoring and comparison of the collected data to monitor progress of the patient's condition.

To this end, the technique of the present invention provides for monitoring and tracking of heart condition. As indicated above, the processing utility 520 is configured and operable for identifying sequence of acoustic signals corresponding to cycle of cardiac activity. Relative changes in time intervals or the amplitudes of heart sounds over time, or abnormalities on heart sounds such as additional sounds, murmur etc., are indicative of progression or regression in heart failure (HF). Such CHF related variation may include:

Reduced intensity of S1 sound—S1 sounds occur at the onset of ventricular contraction and just precede a palpable carotid pulse. The intensity of S1 sounds directly relates to the force of ventricular contraction and the amount of ventricular pressure developed during systole. Lower S1 intensities (amplitude & energy) may commonly be associated with decreased cardiac contractility that may be a result of congestive heart failure, as well as other heart conditions. Additional contributors to reduced, or “soft”, S1 heart sounds may include left ventricular hypertrophy, valvular insufficiencies (mitral), or arrhythmias (AV-block, LBBB) and more. Obesity and emphysema may also reduce the intensity of S1.

Increased intensity of S2 sounds—S2 sounds occur with the end of ventricular systole. S2 sounds are associated with closure of the aortic and pulmonic valves. The intensity of S2 sounds is directly related to the amount of closing pressure in the aorta and pulmonary arteries. In CHF patients, the amplitude and energy of S2 may increase as a result of increased arterial blood pressure (which increases the force in which the pulmonic and aortic valves close).

Decrease or changes in time interval between S1 and S2 heart sounds—The time intervals between an S1 heart sound and an S2 heart sound (ventricular systolic time) following it in a heart cycle may decrease as a result of compromised contractive forces of the ventricles associated with systolic heart failure. This phenomenon is generally attributed to the reduced ability of the ventricles to generate sufficient long-term pressure in order to propel blood to the aorta and pulmonary trunk (especially in patients with reduced ejection fraction).

Presence of S3 heart sounds—The S3 heart sound may appear at the end of the heart cycle. Although presence of S3 sounds may be normal for healthy young adults or children, it is often a sign of heart illness for adults over the age of 40 years. The presence of an S3 heart sound may be indicative of ventricular dysfunction and/or rapid ventricular filling (volume overload), as occurs in CHF patients.

Presence of S4 heart sounds—The S4 heart sound may appear immediately before the normal S1 sound and results from atrial contracting forcefully trying to overcome stiff and hypertrophic ventricle. Presence of S4 sounds has been found to be indicative of hypertension, hypertrophic cardiomyopathy, cardiomyopathies, ischemia and/or myocardial infarction.

Heart rate variability (HRV) outside predetermined limits—Heart rate variability is a measurement of the time variation between heart-beats over certain time frame. HRV data may provide a biomarker data indicating autonomic nervous system functionality. Generally, decreased HRV, being under predetermined lower threshold, is associated with increased sympathetic tone, increased heart rate (HR) and stress. This is while increased HRV, being above a corresponding upper threshold, is associated with increased parasympathetic tone, decreased heart-rate and lower levels of stress. In this respect, lower HRV values may be associated with increased risk for mortality in patients following myocardial infection, ischemic cardiomyopathy and CHF. Moreover, high-risk subgroups identified by HRV may be considered candidates for additional drug therapy and adjusted treatment protocols.

Additional acoustic signal patterns may include existence of murmur sounds, elongated S1 or S2 sounds, as well as additional variations in heart sound patterns as may be determined upon monitoring of patient's heart activity continuously or periodically over time.

As indicated above, the processing utility 520 is generally be operable for analyzing one or more of the above described parameters based on the received data about vibrations, including a sequence of acoustic signals, collected from the subject. Based on the sequence, the processing utility may determine momentary heart rate, variation of the heart rate, amplitude and energy of S1 and S2 heart sounds and presence of S3 and S4 heart sounds, and provide data indicative thereof to an operator, thus indicating of heart condition of the subject. Further, in some configurations, the processing utility 520 may store data of heart activity sounds collected from the subject, and compare earlier (past) data stored in a storage utility (local or remote storage utility) to newly collected heart activity data enabling comparison to identify variations in heart activity and progress or regression in heart condition of the subject.

In some further embodiments, the processing utility may utilize data about additional acoustic signals for enhancing the heart condition data. Such additional data may include:

Pathological lung breathing sounds such as prolonged breathing sounds that are often known as bibasilar rales or “crackles”. These prolonged breathing sounds relate to and increased shortness of breath with increased respiratory rate. This may e.g. result from pulmonary edema that may be secondary to left-sided CHF.

Decreased pulse pressure (PP). Variation and decrease in pulse pressure are considered to be predictors of death in patients with progressive (mild to advanced) HF.

Changes in Carotid-radial pulse wave velocity (PWV). Such PWV changes may occur in CHF patients, although being more closely associated with arterial stiffness.

Some additional parameters may also be provided to the processing utility externals, e.g. manually or utilizing additional measurement units providing corresponding data, may include: jugular venous distension (due to increased right atrial pressure); and weight gain and evidence of pitting edemas in lower extremities (swollen feet) due to failure to sufficiently drain body fluids. Further, as indicated above, such parameters may be compared to previously measured data stored in a storage utility, and compared to current measurements for efficient follow up and detection of changes in heart condition.

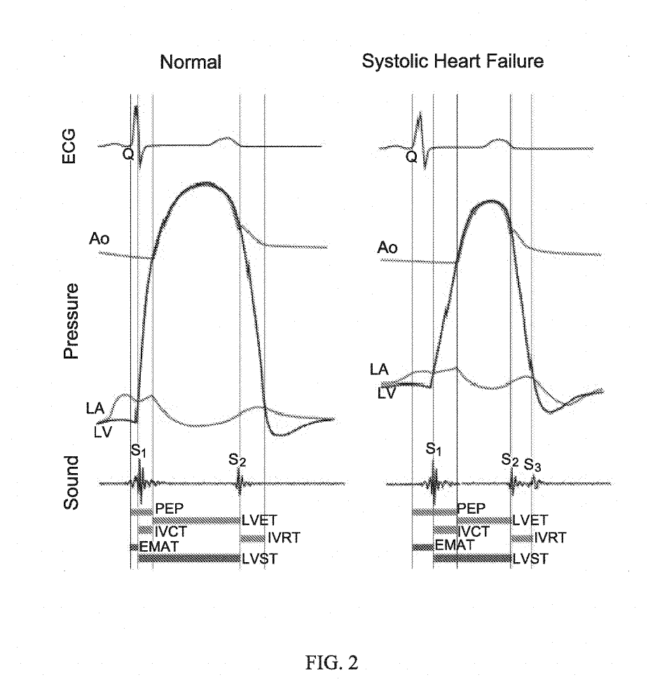

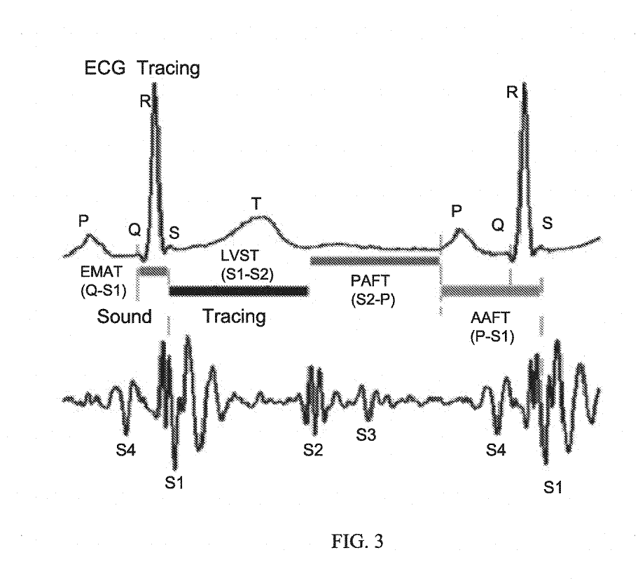

In this connection reference is made to FIG. 2 and FIG. 3 illustrating time variation in electric and acoustic heart activity and exemplifying S1, S2 as well as S3 and S4 heart sounds along a heart pulse cycle. FIG. 2 shows ECG, pressure and sound graphs for a heartbeat period for normal and for a patient with systolic heart failure, and FIG. 3 shows ECG and sound graphs exemplifying heart activity.

As shown in FIG. 2, when the heart ventricles are contracting (Q), an acoustic signal S1 is formed as a result of blood being forced into the arteries. Further, this is associated with increased pressure at the aorta (AO), left ventricle (LV) and reduced pressure at the left atrium (LA). When the ventricles are relaxed, the arterial valves close forming S2 acoustic signal. Looking at operation of a heart suffering from systolic heart failure condition, the time period between acoustic signals S1 and S2, and the corresponding flow variations, may be shortened and a third heart sound S3 may be present resulting from palpitation of blood between the left ventricle and the aorta. FIG. 2 also include timeline information associated with durations of heart activity periods such as: pre-ejection period PEP, left ventricle ejection time LVET, intraventricular conduction time IVCT, Isovolumic relaxation time IVRT, electromechanical activation time EMAT and left ventricular systolic time LVST. Generally, these heart activity periods are well known in the medical community and variation in duration of one or more of the heart activity periods may be indicative of various heart conditions. As indicated above, the present technique utilizes monitoring of acoustic data, using optical measurement and monitoring, for enabling efficient detection and follow-up on heart condition for patients. Accordingly, the heart operation periods are exemplified herein in association with typical heart sounds such as S1, S2 and at times S3 and S4 to emphasize that efficient and reliable monitoring of heart activity sounds may provide valuable data on the patient's heart condition and/or variation thereof.

FIG. 3 shows key landmarks of the ECG including P-Q-R-S-T electric activity and S1, S2, S3 and S4 heart sound recordings in the cardiac cycle and the main acoustic cardio-graphic parameters. As shown, S4 sound may appear at beginning of the cycle and S3 at the end, immediately after the S2 sound. The ECG chart includes P wave corresponding to atrial contraction, Q, S, R waves corresponding to ventricle contraction and T wave corresponding to electrical reset of the heart's activity. Additional notations are: EMAT—electromechanical activation time; IVCT—Isovolumic contraction time; LVST—left ventricular systolic time; LA—left atrium; LV—left ventricle; LVET—left ventricular ejection time; PAFT—pre-atrial filling time; AAFT—accelerated.

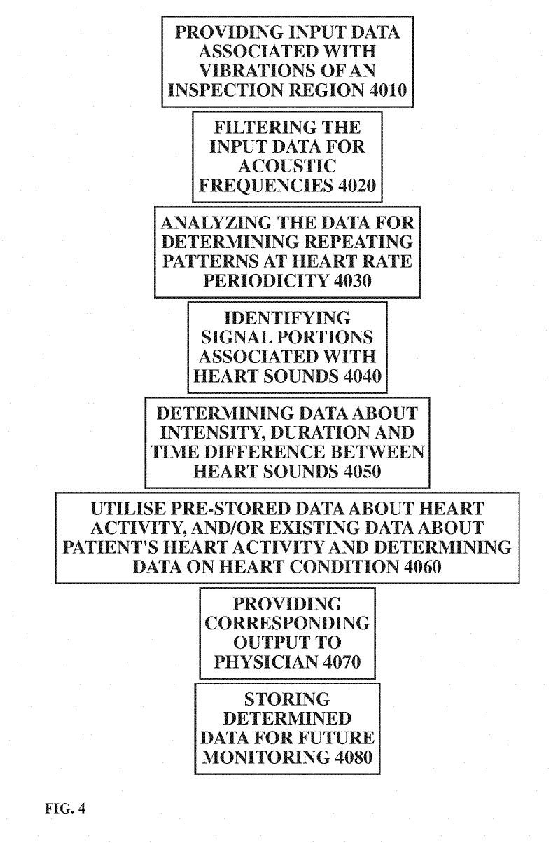

Reference is made to FIG. 4 schematically illustrating operational technique of the processing utility 520 according to some embodiments of the invention. Generally, the technique includes providing, or receiving input data associated with vibrations of an inspection region on a patient's body 4010. The input data is generally determined by determining correlations between sequence of image data pieces associated with speckle patterns collected as described above. The vibration data is preferably filtered in accordance with selected frequency band 4020, e.g. corresponding with acoustic signals such as frequencies between 20 Hz to 20 KHz, however additional frequencies may be used even if higher than 20 KHz. The acoustic data is further processed 4030 for determining repeating patterns corresponding to periodicity of heart activity. Such heart activity periodicity may vary between 30 beats per minute (bpm) to about 220 bpm, and is typically in the range of 60-100 bpm. The repeating patterns are examined 4040 for determining signal portions associated with specific heart sounds as described above, e.g. S1, S2 sounds S3 and/or S4 if exist, and murmur sounds. For the identified sounds, the technique may be operated for determining data about signal intensity and durations, as well as time difference between sounds 4050. Such parameters are described above as being indicative of one or more heart conditions. The determined data about sound parameters is analyzed 4060, e.g. using one or more suitably trained neural networks or in comparison to previously measured and stored data of the patient, for determining data about heart condition of the patient and/or variation thereof. The determined heart condition data may be provided as output data to an operator and/or physician 4070, and may be stored for comparison with future data enabling follow-up on the patient 4080.

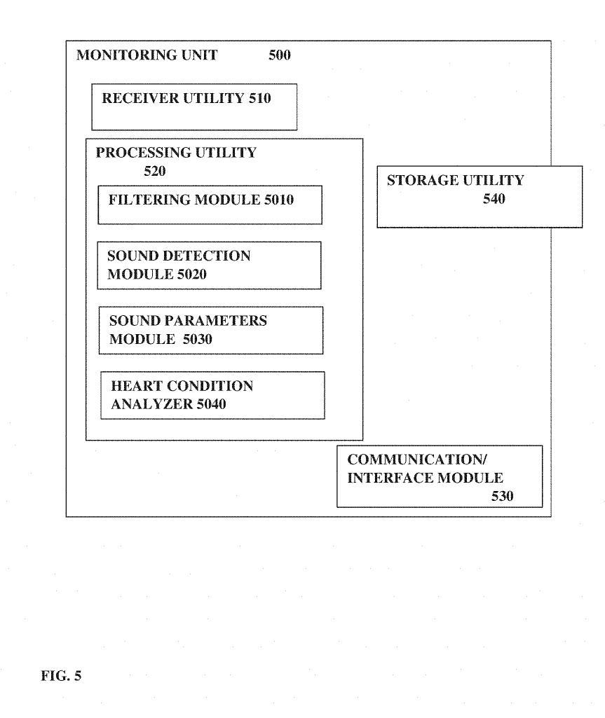

Accordingly, the processing utility 520 and the monitoring unit 500 may include one or more hardware and/or software modules, which are configured and operable for performing the above describe operational tasks. FIG. 5 illustrates schematically configuration of the monitoring unit 500 and its processing utility 520 according to some embodiments of the invention. As shown, and as described above, the monitoring unit typically include certain communication modules such as receiver utility 510 and communication and interface module 530 enabling efficient communication and interface with the one or more optical collection units 100 and operator or physician, as well as include, or be in communication with, one or more storage utilities 540. The receiver utility 510 is configured for receiving input data in the form of sequence of image data pieces and/or corresponding correlation therebetween, and transfer the data to the processing utility 520. As indicated above, the input data may be pre-processed for determining correlation between collected speckle patterns, e.g. by the one or more collection units 100, the receiver utility 510 or the processing utility 520 itself. Further, the collected data and output data indicative of patient's heart condition may preferably be stored in the storage utility 540 for additional processing as well as follow-up on variation is the patient's heart condition.

The processing utility 520 may include filtering module 5010 configured for filtering selected frequency band from the input data; sound detection module 5020 configured for processing the filtered acoustic data and identifying selected heart sounds in accordance with periodicity and repetition thereof; sound parameters module 5030 configured for determining various sound related parameters such as sound amplitude, energy, duration and time difference between sounds as described above with respect to S1, S2, S3 and S4 sounds; and heart condition analyzer 5040 configured for processing the received parameters and determine data about heart condition of the patient.

As also indicated above, the heart condition analyzer 5040 may utilize pre-stored data, e.g. measured heart activity data of the patient from previous check-up pre-stored in the storage utility 540, as well as additional one or more databases for determining variation of heart condition in accordance with changes in patient's state/health (which may also be stored in the storage utility 540). Further, the heart condition analyzer 5040 may be configured as a neural network, trained and configured in accordance with plurality of diagnosed patients and corresponding heart sounds in order to optimize detection of various heart conditions in combination with the above described criteria for heart conditions.

As also described above, the processing utility may be configured for storing data associated with the various determined parameters including acoustic signal stream, identified hear sounds, heart sound parameters and determined data about heart condition of the patient for future use and comparison to enable continuous monitoring of patient's health. It should be noted that the present technique solves one of the major disadvantages of patient diagnosis using acoustic data (e.g. stethoscope) as is provides objective analysis and enables storing of the measured data for additional processing and review, as well as comparing for determining changes in heart conditions.

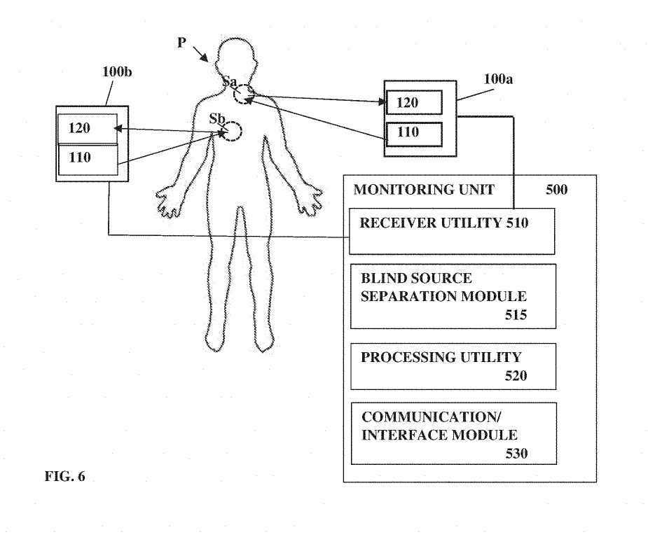

To improve efficiency of heart sound detection, and separation between heart sounds and other acoustic signals that may be associated with subject's activity, the present invention may utilize a plurality of two or more measurement units 100a and 100b. A configuration of a multi-measurement system according to some embodiments of the invention is exemplified in FIG. 6. The different measurement units are connectable to a one or more monitoring system 500 and preferably to a single monitoring system as exemplified in FIG. 6. Each of the measurement units is directed at a different inspection region Sa and Sb on the subject P and configured to collect vibration/acoustic data therefrom. The monitoring unit further includes a blind source separation module 515 configured for receiving data streams of acoustic signals detected by each of the measurement units, and analyze the plurality of data streams by one or more tomographic blind source separation technique.

In this connection, the term tomographic blind source separation generally relates to a group of methods for processing reading of a mixing of a set of discrete number of signals from various directions and performing reconstruction as well as blind source separation of the linearly mixed signals. Each of the measurement units senses nano-vibrations from different directions, e.g. from the front as well as from the back, providing both heart and lung sounds as well as additional sounds that may be collected. Processing the plurality of data streams by blind source separation techniques may separate between noises associated with heart beats noise and breathing sounds, thus enabling analysis of those signals separately (via hardware) and use of the information for early diagnosis of CHF.

Radon transform is often useful in blind source separation providing data about the sources from a plurality of mixed signals. The mathematical definition of the conventional Radon transform is:

g(s, θ)=∫∫f(x, y)δ(x·cos(θ)+y·sin(θ)−s)dxdy

Where g (s, θ) is the Radon transform at all angles θ of a two-dimensional map and s is depth parameter along angle θ direction, f(x,y) and δ is the Dirac delta function.

The blind source separation module is thus configured for receiving a plurality of acoustic data streams and processing the acoustic data streams to determine location source of different acoustic signals, thus enabling to identify heart sounds over lung breathing sounds and any other acoustic signal that might be collected.

It should be noted that in embodiments where the blind source separation is not used, different signals may be separated based on temporal and frequency parameters. For example, heart sound generally have between 40-200 cycles per minute and each cycle includes 2-4 short sounds. This is while breathing sounds are longer and lower in amplitude, and relate to between 4-50 cycles per minute.

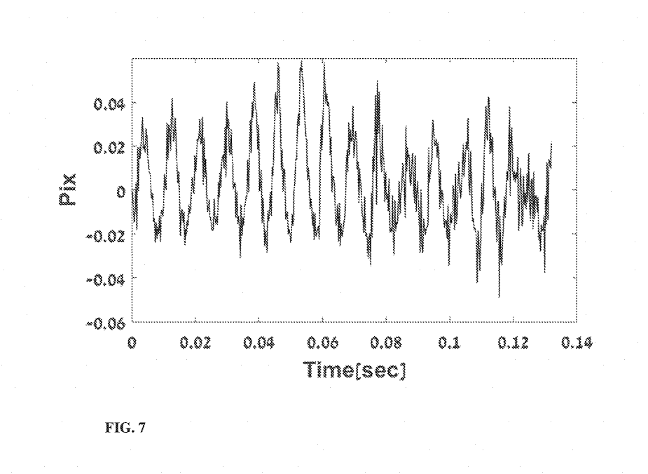

The measurement units 100 are generally configured to emit coherent radiation of a selected wavelength range onto the inspection region and collect data about secondary speckle pattern in the returning radiation. The conventional technique utilizes optical radiation of visible or near IR wavelength, enabling the use of optical arrangement for providing image data. The technique of the present invention may also utilize electromagnetic radiation at longer wavelength, e.g. 1 GHz and up to 3 THz, or between 3 GHz and 300 GHz, providing better penetration depth enabling to determine data indicative of nano-vibrations as well as additional data associated with electrical activity varying scattering properties of tissue with respect to micro-wave radiation. An example of the use of micro-wave speckle detection is exemplified in FIG. 7 illustrating correlation between a plurality of consecutive micro-wave generated speckle patterns collected by inspection of a motor cover. As shown, the motor generates vibrations at frequency of about 12 Hz that are clearly detected by micro-wave speckle correlation. Further, it should be noted that either when using linger wavelength radiation or optical radiation, the monitoring system may also be connected to an ECG unit for providing heart electrical data separately from vibration and acoustic data.

1. A monitoring system for use in monitoring heart condition of a subject, the system comprising:
a receiver utility for receiving input data comprising data corresponding to signals continuously collected from the subject over time and being indicative of a sequence of heart sounds;
a processing utility configured and operable for processing said input data to determine data indicative of heart condition of the subject; said processing comprises: identifying within said input data a sequence of acoustic signals corresponding to heart sounds comprising S1 signals corresponding to ventricular contraction and S2 signals corresponding to end of ventricular systole; analyzing the sequence of acoustic signals in accordance with parameters including signal periodicity, amplitude of S1 and S2 sounds, and intervals between S1 and S2 sounds within the collected sequence; and generating data indicative of cardiac condition of the subject in accordance with the analyzed data.

2. The monitoring system of claim 1, wherein said cardiac condition relates to Congestive Heart Failure (CHF).

3. The monitoring system of claim 1, wherein said analyzing comprises at least one of:
(i) analyzing the amplitude of the signal S1 and determining a measure of force of ventricular contraction and amount of ventricular pressure developed during systole for identifying a condition of decreased cardiac contractility,
(ii) analyzing the amplitude and energy of the signal S2 and determining a measure of amount of closing pressure in an aorta and pulmonary arteries for identifying a condition of increased arterial blood pressure; and
(iii) analyzing the time interval between the signals S1 and S2 of the cycle and determining whether said time interval is shorter than a predetermined value for identifying a condition of systolic heart failure.

4. The monitoring system of claim 1, wherein said processing further comprises analyzing a time pattern of at least two of said cycles for identifying a condition of appearance of a signal associated with a third heart sound S3 appearing at an end of a cycles and being indicative of illness condition.

5. The monitoring system of claim 1, wherein said processing further comprises analyzing a time pattern of at least two of said cycles for identifying a condition of appearance of a signal associated with a fourth heart sound S4 appearing at a beginning of a cycle and being indicative of illness condition.

6. The monitoring system of claim 1, wherein said receiver unit is configured for signal communication with at least one measurement unit operable for continuously collect said acoustic signals from the subject over time and providing said corresponding data to be received by the receiver unit.

7. The monitoring system of claim 6, wherein said measurement unit comprises: at least one radiation source unit configured for generating coherent electromagnetic radiation of a selected frequency range and to direct the generated radiation towards a target; and at least one radiation detection unit associated with said at least one radiation source unit, the at least one radiation detection unit is configured for collecting radiation returning from said target and for generating data indicative of a secondary speckle pattern formed in said collected radiation.

8. The monitoring system of claim 6, wherein said receiver unit being configured and operable for signal communication with a plurality of two or more measurement units, said processing utility being configured and operable for receiving input data corresponding to data received from said plurality of measurement units and for performing blind source separation on said input data to thereby separate heart related acoustic signals from other signals detected by said plurality of measurement units.

9. The monitoring system of claim 8, wherein said plurality of two or more measurement unit is configured such that said plurality of measurement units are directed at a plurality of different inspection regions on said subject.

10. The monitoring unit of claim 6, wherein said least one measurement unit is configured to utilize coherent radiation at a selected frequency range being between 1 GHz and 3 THz.

11. The monitoring unit of claim 10, wherein said selected frequency range is between 2 GHz and 300 GHz.

12. A system comprising:
at least one radiation source unit configured for generating coherent electromagnetic radiation of a selected frequency range and to direct the generated radiation towards a target;
at least one radiation detection unit associated with said at least one radiation source unit, the at least one radiation detection unit is configured for collecting radiation returning from said target and for generating data indicative of a secondary speckle pattern formed in said collected radiation; and
a control unit connectable to said at least one radiation source unit and said at least one radiation detection unit and configured for receiving input data comprising at least one sequence of image data pieces associated with detected secondary speckle patterns and for processing said input data to determine one or more parameters of the target.

13. The system of claim 12, wherein said target being a patient, said one or more parameters of the target comprise one or more parameter of cardiac activity of said patient.

13. The system of claim 12, wherein the control unit comprises at least one processing utility; the processing utility comprises:
a correlation module configured and operable for receiving data about a sequence of input data pieces, each corresponding to a detected speckle pattern, and for processing the sequence of data pieces to determine correlation function between consecutive data pieces, said correlation function being indicative of variation in position of points within an inspected region;
a signal detection module configured for receiving data about variation in position of points within said inspected region and determining therefrom signal data corresponding to selected one or more parameters of the target.

15. The system of claim 14, wherein the signal detection module comprises:
frequency filtering module configured and operable for selectively filtering said data about variation in position of points within an inspected region and determine signal data associated with acoustic signals originated from said inspected region;
cardiac activity module configured and operable for receiving said acoustic signals and determined signal portions associated with cardiac activity sounds;
cardiac efficiency module configured and operable for receiving data about cardiac activity sound from the cardiac activity module and determine one or more parameters of efficiency of cardiac activity.

16. The system of claim 15, wherein said one or more parameters of efficiency of cardiac activity comprise at least one of the following: heart rate, time interval between ventricular and atrial contraction, S3 and S4 cardiac sounds.

17. The system of claim 12, wherein said selected frequency range of electromagnetic radiation comprises radiation frequency between 1 GHz and 3 THz.

18. The system of claim 17, wherein said one or more parameters of the target comprises data indicative of internal electrical activity of the target.

19. The system of claim 12, comprising a plurality of radiation source units configured for directing electromagnetic radiation onto said target from a plurality of different direction and a corresponding plurality of radiation detection unit, said control unit being configured and operable for receiving input data comprising a plurality of data streams corresponding to secondary speckle patterns collected by said plurality of radiation detection units and for processing said input data by blind source separation to thereby determine tomographic data of said target.

20. The system of claim 19, wherein said control unit comprises at least one processing utility; the processing utility comprises:
a correlation module configured and operable for receiving input data about comprises a plurality of sequences of input data pieces, each corresponding to a detected speckle pattern, and for processing the sequence of data pieces to determine correlation function between consecutive data pieces, said correlation function being indicative of variation in position of points within an inspected region;
a blind source separation module configured and operable for receiving a plurality of data pieces each corresponding to correlations between consecutive speckle patterns detected by certain radiation collection unit and for processing said plurality of correlations for determining tomographic data indicative of parameters of the target.

21. The monitoring system of claim 1, wherein said analyzing comprises utilizing intervals between at least S1 and S2 sounds for determining left ventricular systolic time (LVST), thereby providing cardiac activity data corresponding to ECG data.

22. The monitoring system of claim 8, wherein said processing utility being configured and operable performing blind source separation on said input data for separating between noises associated with heart beats noise and breathing sounds.

23. The monitoring system of claim 15, wherein said cardiac activity module is configured and operable for determining heart sounds comprising S1 signals corresponding to ventricular contraction and S2 signals corresponding to end of ventricular systole and for utilizing intervals between at least said S1 and S2 signals for determining left ventricular systolic time (LVST), thereby providing cardiac activity data corresponding to ECG data.

24. The system of claim 15, wherein said frequency filtering module is configured for filtering said data about variation in position of points within an inspected region for obtaining data associated with acoustic signals comprising data varying at frequencies between 20 Hz to 20 KHz.

25. The system of claim 15, wherein said signal portions associated with cardiac activity sounds determined by said cardiac activity module comprise at least one of S1, S2 and murmur cardiac sounds.
